(19) United States
(12) Patent Application Publication
(10) Pub. No.: US 2014/0093946 A1
(43) Pub. Date: Apr. 3, 2014

(54) SYSTEM FOR OPTIMIZING THE INTRODUCTION OF NUCLEIC ACIDS INTO CELLS USING MAGNETIC PARTICLES

(71) Applicant: General Electric Company, Schenectady, NY (US)

(72) Inventors: Jean-Baptiste Mathieu, Clifton Park, NY (US); Brian Michael Davis, Albany, NY (US); Vasile Bogdan Neculaes, Niskayuna, NY (US); Evelina Roxana Loghin, Rexford, NY (US); Kenneth Roger Conway, Clifton Park, NY (US)

(73) Assignee: General Electric Company, Schenectady, NY (US)

(21) Appl. No.: 14/039,323

(22) Filed: Sep. 27, 2013

Related U.S. Application Data
(63) Continuation-in-part of application No. 13/630,970, filed on Sep. 28, 2012, now abandoned.

(51) Int. Cl. C12N 15/64

(52) U.S. Cl. CPC C12N 15/64
USPC 435/285.1

(57) ABSTRACT

An embodiment of a system is provided herein, wherein the system allows for the analysis and selection of numerous experimental conditions to optimize transfection efficiency and cell viability. The system is used for magnetic particle based nucleic acid delivery by optimizing various parameters. The system comprises a control module; an incubation module for incubating magnetic nanoparticle and nucleic acid; a transfection module and an analysis module.

### CROSS REFERENCE TO RELATED APPLICATIONS

This application is a continuation-in-part of U.S. patent application Ser. No. 13/630,970 entitled “System for optimizing the introduction of nucleic acids into cells using magnetic particles”, filed Sep. 28, 2012; which is herein incorporated by reference.

### FIELD OF THE INVENTION

The present disclosure generally relates to a system for optimization of various conditions for the efficient transfection of nucleic acids into cells using magnetic particles.

### BACKGROUND

A variety of methods are known in the art for the introduction (e.g., transfection) of nucleic acids (e.g., DNA, RNA, etc.) into cells. Such methods include both chemical and physical techniques and are accomplished by, for example, electroporation, exposure of cells to liposomes comprising the nucleic acids of interest, the use of viral vectors, and particle-mediated methods such as magnetic delivery. Independent of the method of transfection used to introduce nucleic acids of interest into cells, experimental conditions must be optimized to maximize transfection efficiency. The optimization of these parameters is often extremely time-consuming and labor-intensive.

Magnetic delivery of nucleic acids into cells (also referred to in the literature as “magnetotransfection”) is performed by first mixing/incubating the nucleic acids with magnetic nanoparticles, exposing these nucleic acid/magnetic particle complexes to cells, and applying a magnetic field to enable the entry of the nucleic acid/magnetic complexes into the cells usually through endocytosis. Although magnetic delivery of nucleic acids has been used successfully to transfect cells and commercially available kits for performing this technique, a significant number of experimental parameters must still be optimized to achieve a desirable level of transfection efficiency.

Accordingly, there exists in the art a need for a system to maximize transfection efficiency of magnetic nucleic acid delivery that reduces the time, labor, and financial resources required to maximize transfection efficiency and cell viability when performing this transfection method.

### BRIEF DESCRIPTION

An embodiment of a system is provided herein, wherein the system is for optimizing introduction of a nucleic acid into a cell using magnetic delivery. The system comprises a control module; an incubation module for incubating magnetic nanoparticle and nucleic acid and a transfection module.

An embodiment of a system is provided herein, wherein the system is for optimizing introduction of a nucleic acid into a cell using magnetic delivery. The system comprises a control module; an incubation module for incubating magnetic nanoparticle and nucleic acid; a transfection module and an analysis module.

### DRAWINGS

These and other features, aspects, and advantages of the present invention will become better understood when the following detailed description is read with reference to the accompanying drawings in which like characters represent like parts throughout the drawings, wherein:

FIG. 1 provides a schematic representation of one embodiment of a system for optimizing introduction of nucleic acids into cells by magnetic particle-based transfection of cells.

FIG. 2 provides a schematic representation of one embodiment of a system for optimizing introduction of nucleic acids into cells by magnetic particle-based transfection of cells.

FIG. 3 represents bar graphs showing the higher plasmid transfection efficiency into CHO cells using magnetic nanoparticles compared to the transfection in absence of magnetic nanoparticles.

FIG. 4 represents FITC labeled siRNA delivery into HEK293 cells showing similar transfection efficiency, however different copy number (x-mean intensity) of siRNA using different types of magnetic particles in absence of magnetic field.

FIG. 5 represents similar transfection efficiency of siRNA into B35 cells using magnetic particles of different size with or without magnetic field.

FIG. 6 represents differential plasmid transfection efficiency into NIH3T3 cells in presence or absence of magnetic field.

FIG. 7 represents siRNA transfection efficiency into MEL2 human embryonic stem cells using different temperatures and different exposure time.

FIG. 8 represents siRNA transfection efficiency and cell viability using different types of magnetic particles and magnetic fields, and lipofectamine.

FIG. 9 represents the effect of volume of magnetic nanoparticles in optimization of magnetotransfection to achieve higher plasmid transfection efficiency into B35 cells, without magnetic fields.

FIG. 10 represents the effect of magnetic field amplitude in optimization of magnetotransfection to achieve higher plasmid transfection efficiency into CHO cells, at two concentrations of magnetic nanoparticles.

FIG. 11 represents transfection efficiency for plasmid delivery to HEK293 cells in presence or absence of magnetic fields.

### DETAILED DESCRIPTION

The present invention addresses the limitation of the efficient magnetic delivery of nucleic acids by optimizing the parameters to reduce time and labor required for the entire process. Systems for optimizing the transfection efficiency for introduction of nucleic acids into cells by magnetic delivery are disclosed herein.

To more clearly and concisely describe and point out the subject matter of the claimed invention, the following definitions are provided for specific terms, which are used in the following description and the appended claims. Throughout the specification, exemplification of specific terms should be considered as non-limiting examples.

The singular forms “a”, “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Similarly, “free” may be used in combination with a term, and may include an insubstantial number, or trace amounts while still being considered free of the modified term. Where necessary, ranges have been supplied, and those ranges are inclusive of all sub-ranges there between.

The term “transfection efficiency” refers to the amount of nucleic acid that is successfully introduced into the cells relative to the amount of nucleic acid to which the cells are actually exposed; typically transfection efficiency is defined as the number of cells viable that have been successfully transfected, from the total number of cells. Methods for the determination of transfection efficiency are well known in the art.

“Magnetic delivery of nucleic acids” is intended to refer to the introduction of nucleic acids into cells by mixing and incubating nucleic acids (e.g., DNA, RNA etc.) with magnetic nanoparticles, exposing the resultant nucleic acid/magnetic complexes to cells, applying a magnetic field to the cells in the presence of these complexes to promote their entry into the cells. The phrases “magnetic delivery of nucleic acids”, “magnetic gene transfer”, “magnetic delivery” and “magnetotransfection” may be used interchangeably throughout the specification.

As used herein, the term “nucleic acid” refers to all forms of RNA (e.g., mRNA), DNA (e.g. genomic DNA), as well as recombinant RNA and DNA molecules or analogues of DNA or RNA generated using nucleotide analogues. The nucleic acid molecules may be single stranded or double stranded. Strands may include the coding or non-coding strand. Fragments of nucleic acids of naturally occurring RNA or DNA molecules are encompassed by the present invention. “Fragment” refers to a portion of the nucleic acid (e.g., RNA or DNA). The term “nucleic acid” further includes, but is not limited to, such molecules that are linear, circular, or plasmid in nature. Moreover, the system may be used with small interfering RNA (e.g., siRNA).

As described herein, the magnetic delivery of nucleic acids to any “cell type” refers to any cells capable of up-taking nucleic acids by magnetic delivery. In various embodiments, the cells are referred particularly to mammalian cells, including but not limited to, Chinese Hamster Ovary (CHO) cells, Mesenchymal Stem Cells (MSCs), Embryonic Stem Cells (ESC), Human Embryonic Kidney (HEK) 293cells, NIH 3T3 cells, and B35 cells. This list of cell types is exemplary and not intended to limit the present invention.

An embodiment of a system is provided wherein the system optimizes magnetic particle based nucleic acid delivery by introducing nucleic acids into cells. The system comprises a control module; an incubation module for incubating magnetic nanoparticle and nucleic acid; and a transfection module. In one or more embodiments, the system further comprises an analysis module.

The optimal experimental conditions for performing magnetic delivery of nucleic acids may vary greatly depending on, for example, the cell type and the characteristics of the nucleic acid to be introduced into the cell. Among the experimental parameters that may be optimized include but are not limited to the size, composition, coating, and concentration of the magnetic nanoparticles, the charge of the magnetic particle/DNA complexes, the order of assembly for the magnetic nanoparticle/DNA complexes (i.e., pre-incubation of the DNA with additional chemical vectors such as cationic polymers, before mixing with magnetic nanoparticles), the strength of the magnetic field, the gradient of the magnetic field, the incubation time of the nucleic acid and the magnetic nanoparticle, the nucleic acid concentration, the buffer composition, the pH, the incubation time of nucleic acid with magnetic particles, and the incubation time of the nucleic acid/magnetic nanoparticle complexes with cells prior to and after application of the magnetic field.

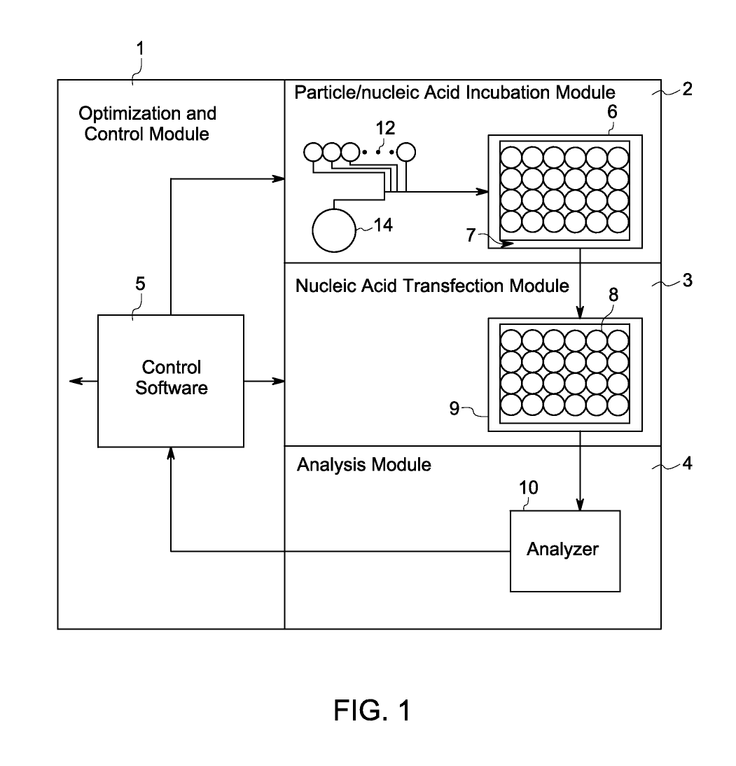

The system for magnetic particle-based transfection is illustrated in FIG. 1, wherein various parameters may be optimized for efficient delivery of nucleic acids into cells. In one embodiment, the system for introduction of a nucleic acid into a cell using magnetic delivery comprises a control module 1, a magnetic nanoparticle and nucleic acid incubation module 2, a transfection module 3. In certain aspects of the invention, the control module 1 comprises control software 5, wherein the control software selects various combinations of experimental conditions to be analyzed for optimizing the magnetic delivery of the nucleic acid. In another embodiment, the system comprises an analysis module 4.

In particular aspects of the instant invention, the system comprises a magnetic nanoparticle and nucleic acid incubation module 2 in which magnetic nanoparticles 12 and nucleic acids 14 are mixed and incubated to form nucleic acid/magnetic nanoparticle complexes. The control software 1 selects a variety of combinations of different properties of the magnetic nanoparticles and the nucleic acid in order to optimize the experimental conditions for magnetic delivery of nucleic acids into cells. With respect to the magnetic nanoparticles such properties include but are not limited to: the concentration of the magnetic nanoparticles, the size of the magnetic nanoparticles, the composition of the magnetic nanoparticles, and the surface properties of the magnetic nanoparticles.

In regard to the different properties of the nucleic acids to be assessed to obtain optimal introduction of nucleic acids into the cells include but are not limited to: the nucleotide sequence of the nucleic acids, the concentration of the nucleic acids, pre-incubation/time of the nucleic acids with additional chemical agents, and the number of nucleotide bases in the nucleic acids. The magnetic nanoparticle and nucleic acid incubation module further comprises a particle incubation reactor, wherein the magnetic nanoparticles and nucleic acids are mixed and incubated for varying times to promote formation of nucleic acid/magnetic nanoparticle complexes under the experimental conditions selected by the control software 1. The particle incubation reactor 6 optionally comprises a platform 7 that permits agitation of the magnetic nanoparticles and nucleic acids during incubation and the formation of the nucleic acid/magnetic nanoparticle complexes. The particle incubation reactor 6 may also comprise a platform 7 that regulates temperature during the formation of the nucleic acid/magnetic nanoparticle complexes.

In certain embodiments of the invention, the system comprises a transfection module 3 comprising a nucleic acid transfection reactor 8 that comprises a plurality of chambers in which cells are mixed and incubated with the nucleic acid/magnetic nanoparticle complexes and exposed to a magnetic field for varying times, at varying magnetic field strengths, and at varying magnetic gradients. The nucleic acid transfection reactor 8 optionally comprises a platform 9 that permits agitation of the cells in the presence of the nucleic acid/magnetic nanoparticle complexes. The nucleic acid transfection reactor 8 may also comprise a platform 9 that regulates temperature during the incubation or the cells and the nucleic acid/magnetic nanoparticle complexes.

In another aspect of the invention, the system for optimizing the introduction of nucleic acids into cells using magnetic particles comprises an analysis module 4 that provides a method for determination of transfection efficiency and cell viability for each combination of experimental conditions tested for the introduction of nucleic acids into the cells. The transfection efficiency and the cell viability data obtained for each set of experimental conditions tested for the introduction of the nucleic acids into the cells is reported to the control software 5 to provide to a user an optimal combination of experimental conditions for introduction of nucleic acids into the cells. In particular uses of the system disclosed herein a range of the experimental conditions is provided to the user for the optimal combination of experimental conditions for introduction of nucleic acids into the cells.

The system may comprise one or more optimization and control modules. This module 1 comprises control software 5, wherein the control software comprises various features. The features may include, but are not limited to: providing commands to the users on experimental conditions to explore the experimental space, drives the experiment, performs an optimization calculation over the data collected, recommends a set of experimental conditions optimized for the user's unique combination of cells, nucleic acids, particles and magnetic generator. The control module may draw recipes from a database of known protocols. In some embodiments, the control module run and control continuous transfection depending on requirement. In one or more embodiments, the control software 5 reports information back to user regarding optimal experimental conditions, measured quantities of transfection efficiency, suggestions for improvements or process control data for production mode.

The particle and nucleic acids, such as DNA or RNA, incubation module 2 comprises a particle incubation reactor 6 comprising magnetic particles 12, wherein the magnetic particles have various particle size, shape or distribution, magnetic materials or surface properties. The nucleic acids 14, such as DNA fragments are combined with the magnetic particles and transferred to the incubation wells of the particle incubation reactor 6. In some embodiments, the particle incubation reactor 6 optionally comprises a platform 7 that permits agitation of the magnetic nanoparticles and nucleic acids during incubation and the formation of the nucleic acid/magnetic nanoparticle complexes. In some embodiments, platform 7 further comprises a heat-plate (heater) along with a temperature sensor that regulates temperature during the formation of the nucleic acid/magnetic nanoparticle complexes. In this module 2, various magnetic particle suspensions are incubated with DNA fragments and the parameters are dictated by the optimization software to optimally scan the experimental space and determine the most efficient parameters for nucleic acid transfection. The various samples may be prepared manually or automatically. Parameters that may be varied include, but are not limited to, particle size distribution, magnetic properties, particle concentration, particle interactions, electrostatic charge, buffer, pH, temperature, agitation, DNA concentration, ligands, and colloidal interactions. Once the optimal parameters have been identified and adjusted, the module 2 may be used to repeatedly and consistently apply the selected recipe in parallel or at larger scale for batch nucleic acid transfection processing.

In some embodiments, as noted, the system comprises a transfection module 3 comprising a nucleic acid transfection reactor 8 that comprises a plurality of chambers in which cells are mixed and incubated with the nucleic acid/magnetic nanoparticle complexes and exposed to a magnetic field for varying times, at varying magnetic field strengths, and at varying magnetic field gradients. In these embodiments, the additional features that may be implemented to the nucleic acid transfection reactor 8 may include, but are not limited to, plate washer, gas inlets for controlled atmosphere, inlets for various chemicals of different properties and charges, thermocycler, micromixer or combinations thereof. In some embodiments, the nucleic acid transfection reactor 8 comprises a platform 9 that permits agitation of the cells in the presence of the nucleic acid/magnetic nanoparticle complexes. The platform 9 may also comprise a heat-pate and a temperature sensor that regulates temperature during the incubation of the cells and the nucleic acid/magnetic nanoparticle complexes. In some embodiments, the platform 9 also comprises one or more magnetic field generator.

In the transfection module 3, the magnetic particle/DNA complexes may be introduced in a container, such as a chamber, a tube, a plate or a vessel containing the cells to be transfected. Parameters are dictated by the optimization software to optimally scan the experimental space and determine the most efficient parameters for nucleic acid transfection. The various samples may be prepared manually or automatically. Parameters that may be varied include, but are not limited to, particle size distribution, magnetic properties, particle concentration, particle interactions, electrostatic charge, buffer, pH, temperature, agitation, DNA concentration, ligands, colloidal interactions, cell type, particle/cell/DNA interactions, time of exposure to magnetic field and combinations thereof. Strength of the magnetic field, geometry, direction of the magnetic field are few parameters that may be varied during optimization of the magnetic field. Once the optimal parameters have been found, the transfection module 3 may be used to repeatedly and consistently apply the selected recipe in parallel or at larger scale, or high throughput, for batch wise nucleic acid transfection processing.

The analysis module 4 comprises a transfection efficiency and cell viability analyzer 10 that provides a method for determination of transfection efficiency and cell viability for each combination of experimental conditions tested for the introduction of nucleic acids into the cells. In embodiments of module 4, the outcome of the transfection operation is analyzed. The analysis systems may include, but are not limited to, flow cytometer, luminometer and spectrophotometer. The transfection efficiency, cell viability and other parameters of interest to describe the transfection may be quantified and the information may be processed by the optimization software to determine the optimal set of experimental conditions. A multistep optimization is possible where, for example, a screening experiment run to evaluate which factors have significant effects. It can then be followed by a coarse optimization experiment that identifies regions of efficient transfection. Finally, a fine optimization experiment finds the optimal conditions within the experimental range identified during the course of experiment. Validation runs may be performed to verify the optimal range prediction. This module may be used for process control while the system is operating in production mode.

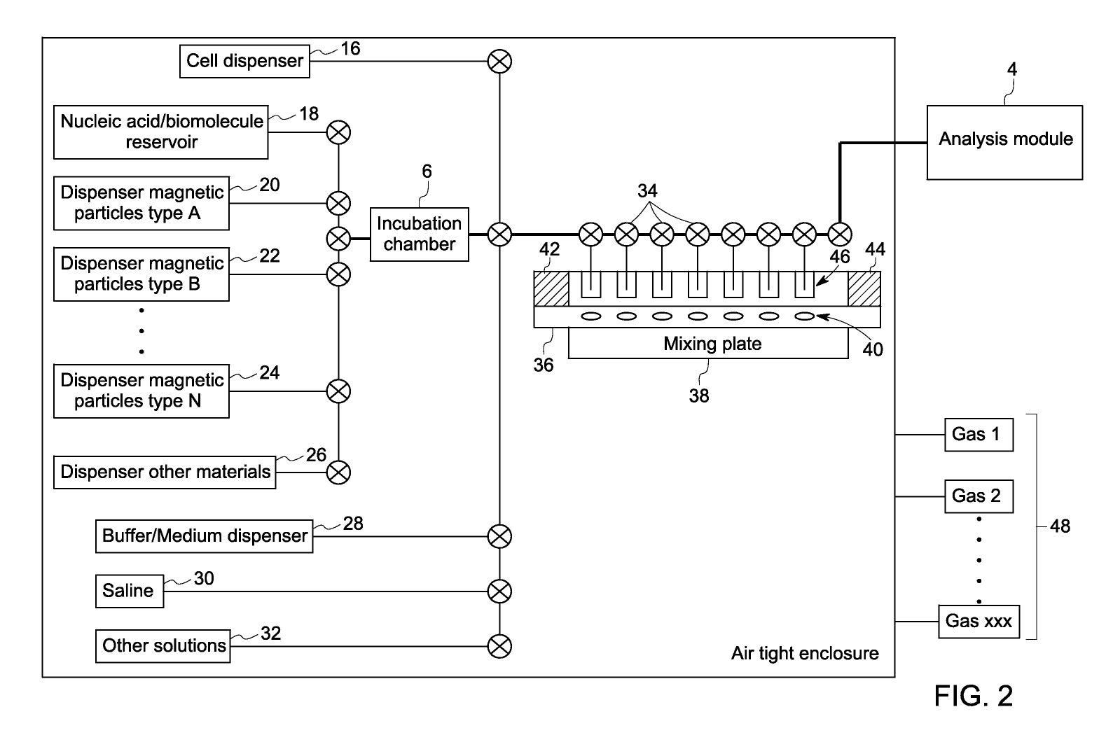

The FIG. 2 illustrates another embodiment of the system, in an exemplary embodiment; it is a microfluidic system, wherein the system comprises an air tight enclosure. The air tight enclosure comprises a cell dispenser 16, which is connected to a channel comprising multiwell plates 46. The system further comprises a nucleic acid/biomolecule reservoir 18 which is connected to an incubation chamber 6 through a valve for controlling the flow of nucleic acids to the incubation chamber. The system further comprises one or more of the magnetic particle dispenser, such as a dispenser 20 for magnetic particles type A, a dispenser 22 for magnetic particles type B, a dispenser 24 for magnetic particle type N. The system may also comprise a dispenser 26 for dispensing other materials, such as a nucleic acid stabilizing reagent, an enzyme or a catalyst. All the dispensers are coupled to the incubation chamber 6 through one or more valves to control the flow of the dispensing material. A buffer or a medium dispenser 28 may be coupled to the channel comprising multi-well plates 46, through one or more valves. The system may comprise a saline flush 30 or chambers for other solutions 32, which are also coupled to the multi-well plates 46. The system comprises transfection module comprising a heat conductive plate 36 below the multi-well plates 46 and comprises an electromagnet array 40. A mixing plate 38 may be present below the multi-well plates for mixing the nucleic acids, magnetic particles with the cells for transfection. The conductive plate 36 further comprises one or more heating and cooling elements 42 and 44. The incubation chamber 6 and the multi-well plates 46 are connected through one or more conduits, and the conduits are open to the multi-well plates through one or more valves 34. An analysis module 4 may be coupled to the transfection module, wherein the analysis module is situated outside the air tight enclosure. One or more chambers 48 comprising one or more gases, such as chamber for gas 1, chamber for gas 2 or chamber for gas xxx are coupled to the air tight enclosure.

In some other embodiments, any step performed by the system may be performed in a manual fashion. In some embodiments, the operation of the system is partially automated, wherein a human intervention is required. In these embodiments, the entry of desired parameters automatically sets-up multiple data points, however, there is a need to operate the system by using a switch, after transfection needs to remove or replace the transfected cells, and load new transfection cells for further transfection. Thus, it would be even more desirable to eliminate such manual intervention and replacement of the transfection cells to save further time and increase operator efficiency.

In one or more embodiments, any step performed by the system may be performed in an automated fashion. To reduce the time-consuming and labor-intensive nature of identifying optimal transfection conditions, the system may ideally be adjusted and performed in an automated fashion as a continuous process. The embodiments, wherein the system is completely automated, the system may run with a single command or a switch, wherein a minimum human intervention is required. In these embodiments, the optimization module sets up the optimization algorithm automatically for the transfection system. The system automatically determines the optimum parameters and controls the system to deliver nucleic acids to the cells.

In one or more embodiments, the system runs in batch production mode. As noted, “batch production mode” refers that the system may run the samples in different batches and then, once the conditions are optimized, it may transfect a batch of samples optimally and performs quality control. The system may apply an optimal combination of experimental conditions for introduction of nucleic acids into the cells. The experimental conditions may be determined by the control software by repeated run of the system and in parallel over a plurality of nucleic acid transfection reactors to increase the throughput of the transfected cells. As noted, the conditions that may be optimized for efficient delivery of nucleic acids may include, but are not limited to, particle size distribution, types of the particles, particle concentration, particle interactions, magnetic field strength, temperature, agitation, concentration of nucleic acids to be delivered, cell types, particle/cell/nucleic acid interactions, time of exposure to magnetic field, buffer composition, pH and combinations thereof.

Additional elements to this system may be alternate magnetic field sources interfaced by the main control software to expand capabilities to special cell types or reagents that are difficult to process with the standard equipment. A magnetic field source may be placed in the module and has a geometry, location and magnetic properties that provide an enhancement of the nucleic acid transfection to the cells. The magnetic field source may be an electromagnet.

The system may be able to perform magnetic nucleic acid delivery and to evaluate the transfection efficiency, including nucleic acid expression and cell viability. In some embodiments, the system may be able to conduct optimization designs of experiments (DOEs) using the samples provided by customers and varying the experimental conditions and levels to optimize transfection efficiency and cell viability.

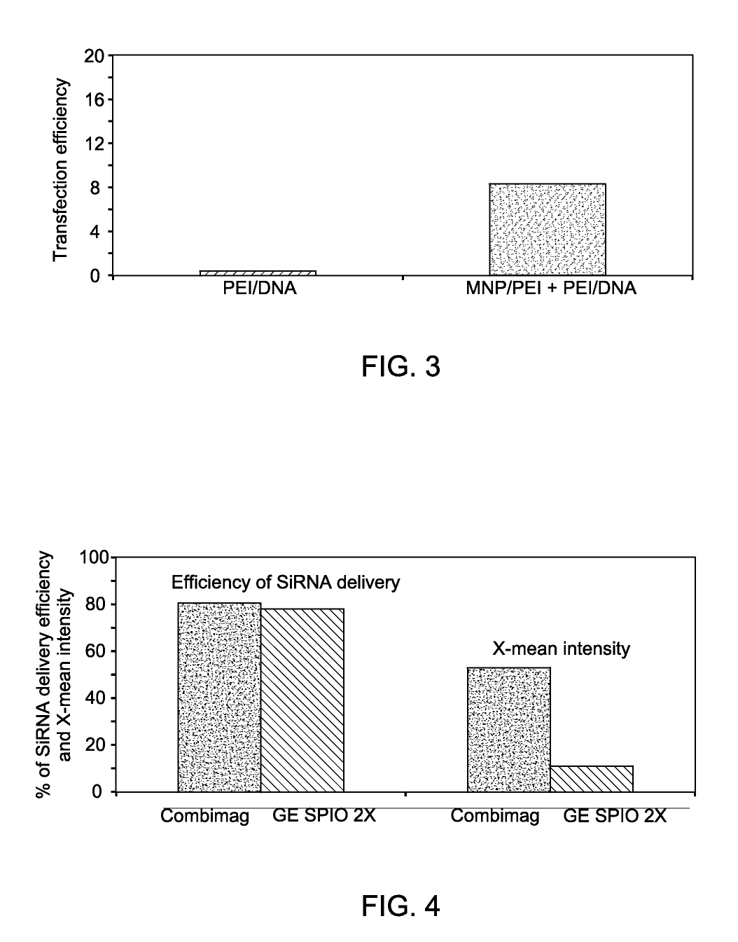

In some embodiments, the presence of magnetic particles during transfection results in significant transfection efficiency compared to absence of magnetic particles. Presence of magnetic particle, such as magnetic nanoparticles (MNP) may trigger the delivery of nucleic acids compared to the case where magnetic particles are not present. For example, the delivery of plasmid DNA to CHO cells in presence of magnetic particles (MNP/PEI+PEI/DNA) is much higher compared to the same in absence of magnetic particles (PEI/DNA), as shown in FIG. 3.

Similarly, the transfection efficiency in presence of two different types of magnetic particles, such as commercially available magnetic particles CombiMag™ and super paramagnetic iron oxide manufactured by GE (GE SPIO) is similar in absence of any magnetic field. For example, the percent of siRNA delivery to HEK 293 cells using two types of magnetic particles, such as CombiMag™ and GE SPIO are the same, whereas the mean fluorescence intensity (X mean) is different for two different samples, without using a magnetic field (correlated to the number of siRNA copies delivered), as shown in FIG. 4. The number of siRNA copies delivered may be optimized by selecting a specific type of magnetic particle, which is also reflected from FIG. 4.

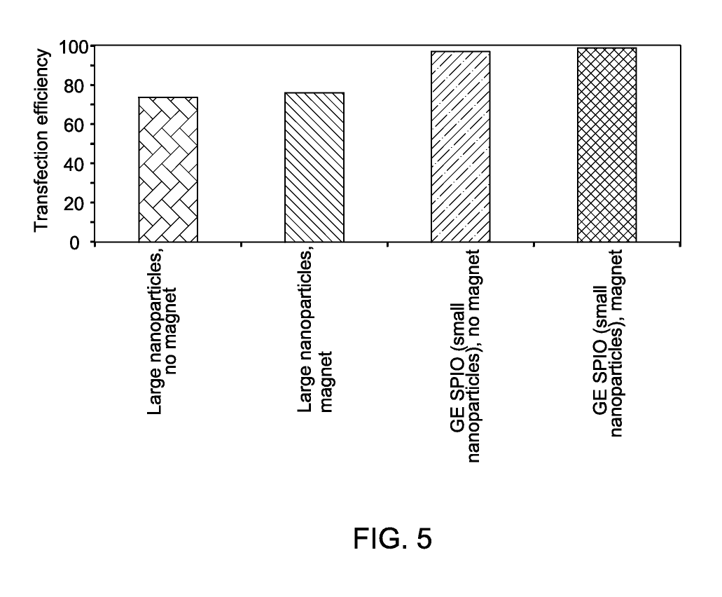

In some embodiments, the particle size affects the transfection of nucleic acids to different cell types. Use of magnetic particles which are varying in size, in presence and absence of magnetic field, may affect the transfection efficiency of the nucleic acids to the cells. The large magnetic particles (120 nm diameter) and small magnetic nanoparticles (15 nm) affect differently to the transfection efficiency of the nucleic acids. For example, the transfection efficiency of siRNA delivery to B35 cells using magnetic particles with different size, such as large magnetic particles and small magnetic nanoparticles with/without magnetic fields, have different effect, as shown in FIG. 5. The efficiency decreases with size of the particles (FIG. 5). The results are same in presence or absence of magnetic field for same particles.

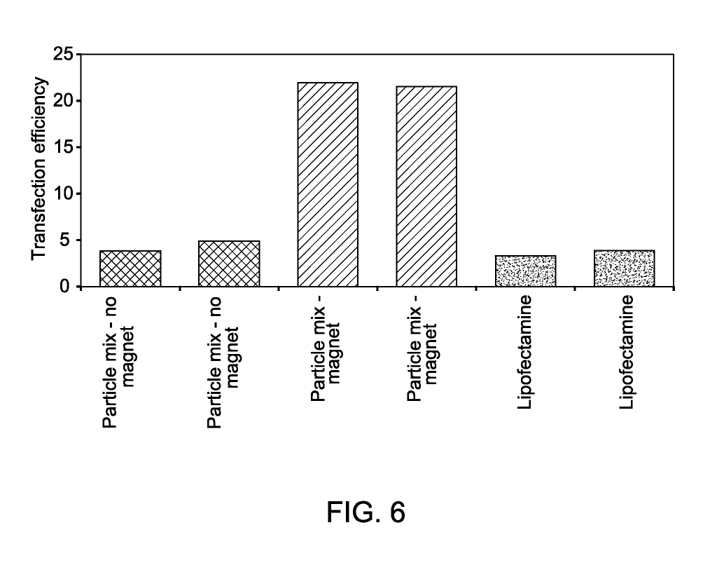

In some embodiments, the presence of magnetic field has substantial effect on magnetic particle mediated transfection (magneto-transfection). For example, the magneto-transfection efficiency for delivery of plasmid DNA to NIH 3T3 cells in presence of magnetic field is higher compared to magneto-transfection in absence of magnetic field, as shown in FIG. 6. The magneto-transfection efficiency using magnetic particles is higher than transfection efficiency using chemical reagents. For example, the transfection efficiency of nucleic acids to NIH 3T3 cells is almost 10 times higher than that using Lipofectamine, as shown in FIG. 6.

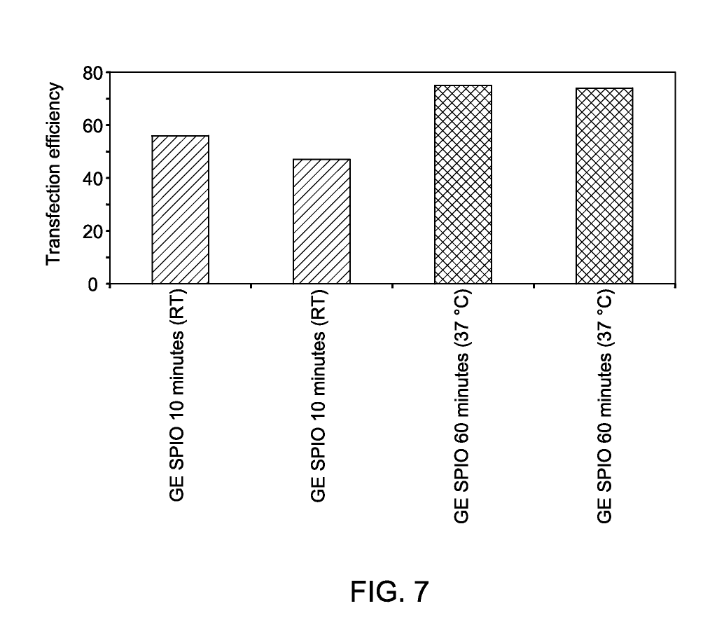

The efficiency of magneto-transfection may be varied at different temperatures, using different exposure time while using the same magnetic particles or same cell types. In some embodiments, the delivery of nucleic acids to cells may be different at different temperatures, such as 10° C., 25° C. or 37° C. In some other embodiments, the delivery of nucleic acids to cells may be different with regard to different exposure time at same or different temperatures. For example, siRNA delivery for human embryonic stem cells (hESC-MEL-2) using GE SPIO magnetic nanoparticles and magnetic field was performed at room temperature (25° C.) for 10 minutes and at 37° C. for 60 minutes, wherein the transfection efficiency of siRNA delivery is higher at 37° C. with exposure time of 60 minutes, as shown in FIG. 7.

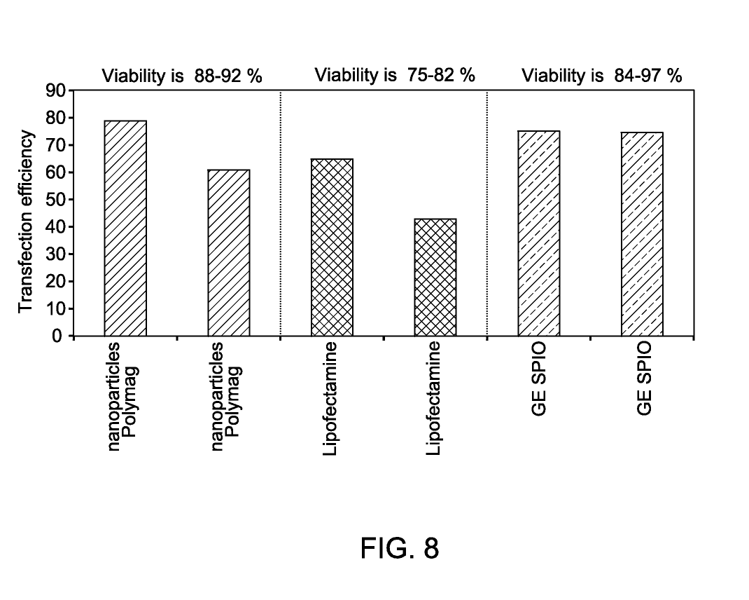

In one or more embodiments, the cell viability may alter using different transfection techniques, such as using chemical reagent or using magnetic particle. For example, siRNA delivery to human embryonic stem cells (hESC-MEL-2) using two types of magnetic particles and Lipofectamine were performed; wherein the viability of cells was measured, as shown in FIG. 8. The data (FIG. 8) shows better cell viability, which is about 85-97% for delivery with GE SPIO magnetic particles for 60 minutes exposure to magnetic fields at 37° C. compared to transfection using Lipofectamine. The transfection using commercially available PolyMag™ nanoparticles also show better viability than Lipofectamine.

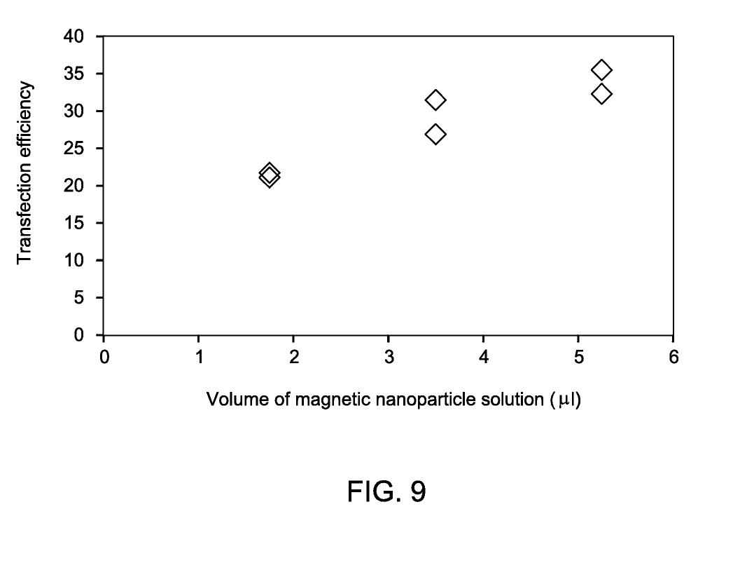

Use of varying concentration of magnetic particles in absence or presence of a magnetic field affects transfection efficiency. The increase of the magnetic particle solution volume in absence of magnetic field increases the concentration of the magnetic particles, which further increases the efficiency of the plasmid delivery, as shown in FIG. 9. For example, varying magnetic particle solution volume under no magnetic field affects plasmid delivery to B35 cells, wherein the efficiency of plasmid delivery increases with increasing the concentration of magnetic particles (FIG. 9). The concentration of magnetic particles or magnetic field strength may affect differently to different cell lines.

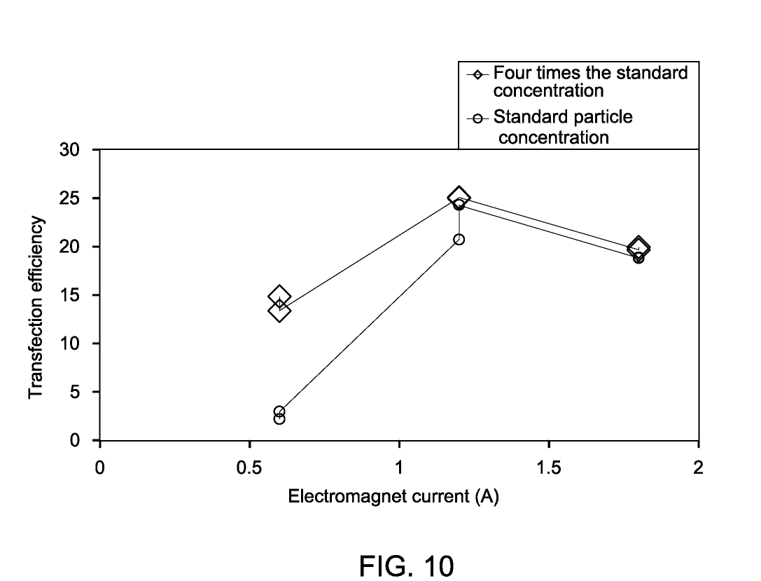

The optimization of magnetic field amplitude for efficient magneto-transfection may be highly desirable. Effects of varying magnetic particle concentration and magnetic field amplitude on transfection efficiency may be significant. The higher transfection efficiency is achieved on optimization of magnetic field amplitude at lower electromagnetic current, as shown in FIG. 10, where the magnet is an electromagnet, and the variation of the magnetic field amplitude is performed by variation of the electromagnet current. The graphs also illustrate how the particle concentration and magnetic field amplitude are related to optimize transfection efficiency. At the lower electromagnetic current setting, higher particle concentration results in higher transfection efficiency; however, at higher electromagnetic settings (higher magnetic field amplitudes), the increased particle concentration does not have much effect on transfection efficiencies.

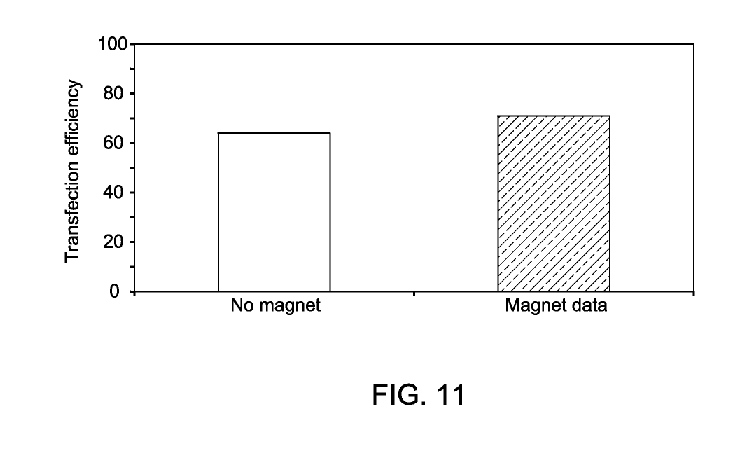

In some embodiments, the system allows magnetic particle based nucleic acid delivery to cells in presence or absence of magnetic field. For example, plasmid DNA delivery to HEK 293 cells using magnetic nanoparticles with and without magnetic fields show almost similar transfection efficiency, as shown in FIG. 11. Various data represents the fact that substantial transfection efficiency is achieved without a magnetic field on different cell types. This may be due to the fact that some of the cells may require a magnetic field with optimized delivery parameters, for some other cells magnetic field may not be beneficial as they may take up the particles passively.

In some embodiments, the system allows either in process or off-line evaluation of various parameters, such as expression level of the nucleic acid, nucleic acid copy number or gene knock down using fluorescence, luminescence, optical density or other methods known in the art. Knockdown of a target gene, copy number of the nucleic acid or protein, and expression level of the delivered nucleic acid may be determined by a direct or indirect measurement of the delivered nucleic acid.

As noted, in some embodiments, the system comprises a detector for measuring expression level of the delivered nucleic acids. The system may further comprise a detector for measuring a copy number of the transfected nucleic acids. In an exemplary embodiment, a direct measurement may include a fluorescent molecule conjugated to the nucleic acid, e.g., a FITC conjugated siRNA. In an alternative embodiment, the nucleic acid that expresses a protein which may be quantified, e.g. a fluorescent or luminescent gene that expresses green fluorescent protein or luciferase may be used to determine an expression level of the delivered nucleic acids. The measurements may determine the percentage of cells that have taken up the nucleic acid, the relative copy number based on the intensity of the signal or combinations thereof.

In one or more embodiments, the system further comprises a detector for measuring a percent of knockdown targeted gene. A gene knockdown event for a target gene caused by the delivery of the specific nucleic acid may be measured indirectly, e.g. delivery of a siRNA sequence to a cell that knocks down or terminate the expression of a target gene may be achieved using the system. In some embodiments, a siRNA knocks down the target gene which expresses a marker protein, for example, green fluorescent protein or luciferase, wherein reduction in the expression of the gene is measurable after delivery of siRNA. An example of off-line analysis is: to transfer the multi-well plate onto a conventional luminometer, fluorimeter, microscope or flow cytometer to assess the various parameters.

In one or more embodiments, the system is used for high throughput applications. The optimization of parameters is significant for molecular delivery into cells to reduce the labor and time as required for the current processes. The system may allow utilization of multiwell plates (e.g, 24 well or 96 well plates) and may be automated for high throughput evaluation of many variables simultaneously or in series. The variables include, but are not limited to, particle size, particle concentration, temperature, macromolecule (such as nucleic acids, proteins) concentration, pre-incubation time, exposure time on cells, pH, buffer/medium composition and magnetic field strength. The output of the system may provide users an optimized set of parameters for maximum efficiency and highest cellular viability.

Following the determination of optimized parameters at high throughput scale, the system allows scale-up of the process of efficient transfection using magnetic particles followed by gene or protein expression analysis. The system may be used to evaluate scalability of a process optimized at 96 well scale, 24 well scale and 6 well scale, leading to larger batches with sufficient cell numbers required for practical applications, for example in bioprocessing or cell therapy applications.

In some embodiments, the fluidic system is designed as high throughput system, wherein a module may be integrated to the system where the reagents may be placed in contact with the cells continuously or sequentially. In one embodiment, the nucleic acids and cells are mixed in a flowing channel. In this embodiment, the cells may move along the channel. In some other embodiments, the reagents may include magnetic particles, fragments of genetic material, chemicals, or surfactants, which are introduced in the flow chamber in a way prescribed by the system to optimize the efficiency of the transfection.

The system may further comprise a temperature sensor and set up arrangement for probing required temperature selecting from a multiple temperatures. In one or more embodiments, the system allows comparison of temperatures through the use of multiple heating and cooling elements adjacent to a conductive plate holder. In these embodiments, the system may establish a temperature gradient across the plate by controlling the elements to provide different temperatures. Individual wells may experience a defined temperature in which efficiency of molecular delivery may be measured. In one or more embodiments, the system further comprises a temperature feedback mechanism for controlling the temperature of each of the well, modifying the temperature depending on user need.

The internal atmosphere of the system, such as each of the wells may be controlled by using various gases such as CO₂, N₂ to name a few which may allow control over the oxygen levels and pH or other physical or chemical parameters that may influence in such a manner. In some embodiments, the internal atmosphere is maintained as CO₂ atmosphere.

The types of molecules that are delivered are typically nucleic acids or proteins, but may also pertain to small molecules that are typically impermeable to a cell membrane in the absence of an active delivery process. The systems for optimizing magnetic delivery of nucleic acids disclosed herein may be used with nucleic acids in any form. The skilled artisan would appreciate that the term “nucleic acid” refers to all forms of RNA (e.g., mRNA), DNA (e.g. genomic DNA), as well as recombinant RNA and DNA molecules or analogues of DNA or RNA generated using nucleotide analogues. The nucleic acid molecules can be single stranded or double stranded. Strands can include the coding or non-coding strand. Fragments of nucleic acids of naturally occurring RNA or DNA molecules are encompassed by the present invention. “Fragment” refers to a portion of the nucleic acid (e.g., RNA or DNA). The term “nucleic acid” further includes, but is not limited to, such molecules that are linear, circular, or plasmid in nature. Moreover, the instant system may be used with small interfering RNA (e.g., siRNA).

The systems described here may be used to optimize the magnetic delivery of nucleic acids to any cell type, particularly mammalian cells, including but not limited to, Chinese hamster ovary (CHO) cells, mesenchymal stem cells (MSCs), embryonic stem cells, human embryonic kidney (HEK) 293cells, NIH 3T3 cells, and B35 cells. This list of cell types is exemplary and not intended to limit the present invention.

In one or more embodiments, the system is designed to minimize cell death, while ensuring highly efficient delivery of nucleic acids. The system employs a suitable buffer to transfect nucleic acids to any mammalian cell line, including primary and difficult-to-transfect cells. The magnetic nucleic acid delivery may ensure minimum cell death or impairment as this process does not employ any chemical reagents having adverse effect on the living system.

Due to the inherent complexity of the magnetic transfection phenomenon, including involvement of multiple factors, the users may not have the resources or skills required to optimize the efficient transfection process. Optimized experimental conditions may be achieved in one day with minimal hands-on time using this device, whereas the approaches presently known in the art are labor intensive and may require multiple experiments over weeks.

In practice, the user may load the reagents (cells, DNA, particles, etc) in separate containers in the system. The system may prepare the samples to run a sample, run the experiments, analyze the results and report back to the customers with the optimized nucleic acid transfection recipe for their particular combination of reagents. In detail, the user inserts particles, DNA, cells in separate chambers of the system. The system prepares various samples with different combinations of experimental conditions based on suitable quality control tool. The magnetic particles combined to the DNA are added in presence of the cells in a magnetic chamber able to vary the magnetic field and magnetic field gradient in alternate current (AC) or direct current (DC) mode. Being a high throughput system, it may analyze several samples in parallel in several magnet chambers to provide different experimental conditions simultaneously. After all samples are tested, the machine may perform an optimization calculation over the data collected and may recommend a set of experimental conditions optimized for the customers unique combination of cells, nucleic acids, particles and magnet. To draw recipes from a database of known protocols may also be possible. The system directs the customer towards the magnetic transfection separation method/tool best suited to use for best results. The user may validate the prediction using the parameters prescribed and the magneto-transfection system of their choice and the analysis system may verify the data, fine tune the protocol and provide quality control. All these steps may be automated if the system handles the transfection itself or if it controls a third party magnetic transfection system.

Some embodiments of the system may bring tremendous value to the customer by providing rigorous experimental control to optimize the process to achieve desired transfection efficiency. A low cost version of the system may allow the user to prepare the samples and run the experiments to generate high quality results.

### EXAMPLES

### Example 1

### Use of Magnetic Particles in Addition to PEI Increases Transfection Efficiency of Plasmid GFP into CHO Cells

Materials:

Chinese Hamster Ovary (CHO, cat# CCL-61) cells were purchased from ATCC®, Manssas, Va., USA. F-12K Medium was purchased from ATCC® (cat#30-2004). Polyethylenimine (PEI), branched average molecular weight 25,000 was purchased from Sigma-Aldrich (cat 408727). PEI was diluted in water to generate a working stock of 80 ug/ml. To perform the transfections, a plasmid encoding maxGFP, a green fluorescent protein from the copepod Pontellina p was used from a kit available from Lonza (cat# VSC-1001). pMaxGFP stock was at 1 μg/μl. GE nanoparticles of 50 nm size were used for this experiment.

Cells and Media:

CHO cells were cultured in cultured in ATCC®-formulated F-12K Medium (cat#30-2004) supplemented with 10% Fetal Bovine Serum (FBS). FBS was purchased from ATCC® (cat#30-2020). Trypsin-EDTA Solution, 1× was purchased from ATCC® (cat#30-2101).

Delivery of pGFP into CHO Cells Using PEI and a Mix of Magnetic Particles/PEI:

Chinese Hamster Ovary (CHO, cat# CCL-61) were cultured in ATCC®-formulated F-12K Medium (cat#30-2004) supplemented with 10% FBS. CHO cells were seeded in 24-well TCP at 0.6×10̂5 cells/well in the recommended media. 24 h post seeding cells were transfected with 1 μg plasmid MaxGFP according to the following protocol. For the magnetic particles (MNP)/PEI+PEI/DNA samples (bar on the right) 25 ul of PEI (40 ug/ml concentration) was mixed with 250 pMaxGFP in dropwise manner (40 μg/ml concentration) and incubated at RT for 30 min to generate PEI/DNA mix. That corresponds to 1 μg pMaxGFP per sample. To generate the MNP/PEI complex 500 MNP were mixed with 500 PEI (40 ug/ml). MNP/PEI (50 nm) were added to the PEI/DNA at a ratio of nanoparticles:DNA of 1.2:1 and allowed to incubate at RT for 1 h. For the PEI/DNA samples (bar on the left) 500 PEI at 40 ug/ml concentration was mixed with 500 pMaxGFP (40 ug/ml) in a dropwise manner and allowed to incubate at RT for 30 min. Each condition was done in duplicates and a master mix was generated. The PEI/DNA and the MNP/PEI+PEI/DNA complexes were then added to the cells and allowed to incubate for ˜24 h. Cells were evaluated for transfection efficiency next day (˜24 h) by flow cytometry using a Beckman Coulter FC500 Flow Cytometer. Briefly, spent media was removed and cells were washed once in PBS at RT. After PBS removal, enzymatic release of the cells was performed using Trypsin-EDTA (ATCC® cat#30-2101) at 37° C. for 2-5 min. Cells were spun down and the cell pellet was resuspended in 1500 PBS and analyzed by flow cytometry in FL1 channel (corresponding to the 525 nm wavelength) according to the protocol. The transfection efficiency for the CHO cells using PEI/DNA in presence of PEI/magnetic nanoparticles is much higher compared to the transfection in absence of magnetic nanoparticles, as shown in FIG. 3. The presence of magnetic nanoparticles (MPN) triggers the plasmid delivery compared to the case where magnetic nanoparticles are not present.

### Example 2

### Different siRNA Copy Number (X-Mean Intensities) Achieved when Different Nanoparticles are Used for siRNA-FITC Delivery into HEK-293 Cells

Materials Used:

Human embryonic kidney (HEK293) cells were purchased from ATCC®, Manssas, Va., USA (cat# CRL-1573). CombiMag particles were purchased from Boca Scientific Boca Raton, Fla., USA (cat# CM20100). Lipofectamine® 2000 transfection reagent was purchased from Invitrogen® (cat#11668019). GE nanoparticles (50 nm) were used at 2× concentration relative to the commercial magnetic particles. Control siRNA FITC was purchased from Santa Cruz Biotechnology (cat# sc-36869).

Cells and Media:

Human embryonic kidney (HEK293) cells were purchased from ATCC®, Manssas, Va., USA (cat# CRL-1573). HEK 293 cells were cultured in ATCC-formulated Eagle's Minimum Essential Medium, (catalog #30-2003) supplemented with 10% FBS.). FBS was purchased from ATCC (cat#30-2020). Trypsin-EDTA Solution, 1× was purchased from ATCC® (cat#30-2101). Cells were cultured according to the manufacturer's protocol.

siRNA-FITC Delivery into HEK293 Using Commercial and SPIO Nanoparticles:

HEK293 cells were seeded at 0.7×10̂5 cells/well in a 24-well TCP. 24 h post seeding cells were used for siRNA FITC delivery as following. For half of the samples CombiMag was used as the vehicle for the siRNA delivery. 1 μl CombiMag and 2 μl Lipofectamine® were used per sample according to the following protocol: 1 μl CombiMag was first mixed with 20 pmoles siRNA-FITC and the complex was allowed to incubate for few minutes. 20 Lipofectamine®2000 was added to the CombiMag/siRNA mix and allowed to further incubate at RT for 30 min. The siRNA/Lipid/CombiMag complexes were added to the HEK293 cells and allowed to incubate with the cells for ˜24 h at 37° C. 5% CO₂. For the rest of the samples GE nanoparticles were used at 2× the concentration of the commercial nanoparticles. The GE nanoparticles were mixed with 20 pmoles siRNA and allowed to sit at RT for 30 min. They were then added to the cells and allowed to incubate for ˜24 h. Cells were evaluated for transfection efficiency next day (˜24 h) by flow cytometry using a Beckman Coulter FC500 Flow Cytometer. Briefly, spent media was removed and cells were washed once in PBS at RT. After PBS removal, enzymatic release of the cells was performed using Trypsin-EDTA (ATCC® cat#30-2101) at 37° C. for 2-5 min. Cells were spun down and the cell pellet was resuspended in 1500 PBS and analyzed by flow cytometry in FL1 channel (corresponding to the 525 nm wavelength) according to the protocol. The SiRNA delivery to HEK 293 cells in presence of two different types of magnetic particles in absence of magnetic field was performed. The data shows transfection efficiency for delivery of siRNA to HEK 293 cells in presence of two different types of magnetic particles, CombiMag and GE SPIO are comparable, wherein the X-mean intensity, which is correlated to the number of siRNA copies delivered, is different, as shown in FIG. 4.

### Example 3

### Variable Effect of the Magnet on siRNA Delivery into B35 Neuroblastoma Cells

Materials:

The B35 rat neuroblastoma cells (cat# CRL-2754) were purchased from ATCC®, Manssas, Va., USA. NeuroMag particles (cat# NM50200) were purchased from Boca Scientific Boca Raton, Fla., USA. To perform siRNA delivery, BLOCK-iT™ Fluorescent Oligo (siRNA-FITC) was used to estimate nucleic acid delivery efficiency (Life Technologies, cat#13750062). The superparamagnetic iron oxide particles (GE SPIO) were generated in-house (GE).

Cells and Medium:

B35 rat neuroblastoma cells (ATCC®, cat# CRL-2754) were cultured in ATCC® formulated Dulbecco's Modified Eagle's Medium (DMEM, cat#30-2002) supplemented with 10% FBS according to the manufacturer's ATCC® protocol.

B35 neuroblastoma cells were seeded in 24 well plates at 0.7×10⁵ cells/well. Cells were allowed to incubate at 37 C overnight and transfected 24 h post seeding at ˜60-70% confluence. Cell count was measured using Countess® Automated Cell Counter (Invitrogen) according to the manufacturer's protocol. Each well was transfected with 20 pmoles of fluorescein isothiocyanate (FITC) conjugated siRNA. 3.50 commercial nanoparticles (NeuroMag) or GE SPIO nanoparticles were used for each well. The delivery was done according to the manufacturer's protocol. Briefly, the NeuroMag particles were mixed with the siRNA and incubated for 15-20 min at RT. The nanoparticles/siRNA complexes were then added to the cells. Half of the samples were incubated on the permanent magnet for 15 min in the 37 C/5% CO₂ incubator and half of the samples were directly incubated in the 37° C./5% CO₂ incubator (no magnet exposure). Each condition was done in duplicate. B35 cells were evaluated for transfection efficiency next day (˜24 h) by flow cytometry using a Beckman Coulter FC500 Flow Cytometer. Briefly, spent media was removed and cells were washed once in PBS at RT. After PBS removal, enzymatic release of the cells was performed using Trypsin-EDTA (ATCC® cat#30-2101) at 37 C for 2-5 min. Cells were spun down and the cell pellet was resuspended in 1500 PBS and analyzed by flow cytometry in FL1 channel (corresponding to the 525 nm wavelength) according to the protocol.

The NeuroMag particles having diameter of 120 nm were selected; which were comparatively larger than the small GE SPIO nanoparticles with diameter of 15 nm. The NeuroMag particles and SPIO magnetic nanoparticles affect differently to the transfection efficiency of the nucleic acids. For example, the transfection efficiency of siRNA delivery to B35 cells using magnetic particles with different size, such as NeuroMag particles and SPIO nanoparticles with/without magnetic fields, have different effect, as shown in FIG. 5. The efficiency decreases with size of the particles (FIG. 5). The results are same in presence or absence of magnetic field for same particles.

### Example 4

### Effect of GE Electromagnet on Plasmid GFP Delivery into NIH3T3 Cells Using Commercial Nanoparticles (CombiMag)

Materials:

The NIH/3T3 mouse fibroblast cells (cat# CRL-1658) were purchased from ATCC®, Manssas, Va., USA. CombiMag particles were purchased from Boca Scientific Boca Raton, Fla., USA (cat# CM20100). Lipofectamine® 2000 transfection reagent was purchased from Invitrogen® (cat#11668019). To perform the transfections a plasmid encoding maxGFP, a green fluorescent protein from the copepod Pontellina p was used from a kit available from Lonza (catalog #VSC-1001). pMaxGFP stock was at 0.5 μg/μl.

Cells and Medium:

NIH/3T3 mouse fibroblast cells (ATCC® cat# CRL-1658) were cultured in ATCC-formulated Dulbecco's Modified Eagle's Medium, (ATCC, cat#. 30-2002) supplemented with 10% FBS according to the manufacturer's protocol.

Plasmid GFP Transfection Using Commercial Particles and Electromagnet in NIH/3T3 Cells:

NIH/3T3 cells were seeded in 24-well TCP at 0.7×10⁵ cells/well. Cells were allowed to incubate at 37° C. overnight and transfected 24 h post seeding at ˜60-70% confluence. Cell count was measured using Countess® Automated Cell Counter (Invitrogen) according to the manufacturer's protocol. Each well was transfected with 0.5 ug pMaxGFP. Experiment was done using two replicates per each experimental condition, which are both represented in the FIG. 6. The experimental samples were transfected with 1 μl CombiMag and 20 Lipofectamine® per sample according to the manufacturer's protocol: 1 μl CombiMag was first mixed with 0.50 pMaxGFP and allowed to incubate for a few minutes. 20 Lipofectamine®2000 was added to the CombiMag/DNA mix and allowed to further incubate at RT for 30 min. The DNA/Lipid/CombiMag complexes were added to the NIH/3T3 cells and allowed to incubate with the cells directly (particle mix no magnet) or placed on the electromagnet wherein the electromagnet used for this test creates a field of about 0.23 T at 1.8 A for another 20 min (particle mix magnet). All samples were incubated overnight in the 37° C., 5% CO₂ incubator for ˜24 h. Two samples were transfected using Lipofectamine®2000 according to the Invitrogen® protocol to serve as transfection controls. Briefly, the DNA-lipid complex was prepared by adding 2 μl Lipofectamine®2000 to 0.5 μg plasmid GFP. The complex was allowed to form by incubating at RT for 15 min. The DNA/lipid was added then to the cells and the transfection was evaluated 24 h later by flow cytometry using a Beckman Coulter FC500 Flow Cytometer. Briefly, spent media was removed and cells were washed once in PBS at RT. After PBS removal, enzymatic release of the cells was performed using Trypsin-EDTA (ATCC® cat#30-2101) at 37 C for 2-5 min. Cells were spun down and the cell pellet was resuspended in 1500 PBS and analyzed by flow cytometry in FL1 channel (corresponding to the 525 nm wavelength) according to the protocol. FIG. 6 shows much higher transfection efficiency in presence of electromagnet compared to no magnet using CombiMag nanoparticles for NIH 3T3 cells.

### Example 5

### Effect of Exposure Time and Temperature on Nucleic Acid Delivery in Human Embryonic Stem Cells Using SPIO Nanoparticles

Materials: MEL-2 human embryonic stem cells were purchased from Millipore® (cat# SCC021). Matrigel™ (cat#356234) was purchased from BD®. mTESR™1 cell culture media was purchased from STEMCELL™ Technologies (cat#05850). StemPro® Accutase® Cell Dissociation Reagent was procured from Life Technologies (cat# A1110501). Y-27632 (ROCK inhibitor) was obtained from Enzo®Life Sciences (cat# ALX-270-333-M001). BLOCK-iT™ Fluorescent Oligo (siRNA-FITC) was used to estimate the transfection efficiency (Life Technologies, cat#13750062). The GE SPIO magnetic nanoparticles were generated in-house (GE). The magnetic field exposure was performed using a commercial magnetic plate (Boca Scientific, cat# MF10000).

Cells and Medium:

MEL-2 human embryonic stem cells (Millipore®, cat# SCC021) are cultured in Matrigel™ coated plates according to the manufacturer's protocol. Cells are maintained in complete mTESR™1 media that is reconstituted according to the manufacturer's protocol. The complete media was generated by adding the mTESR™1 5× Supplement bottle to the mTESR™1 Basal Medium.

siRNA-FITC Delivery Using Magnetic Nanoparticles (GE SPIO) and Commercial Magnet:

MEL-2 human embryonic stem cells were seeded in Matrigel™ coated 24-well plates in mTESR™1 media at 240,000 cells/well. Two sets of conditions were tested: incubation of the cells/siRNA/nanoparticles on the magnet for 10 min at RT and incubation on the magnet at 37° C., for 60 minutes. For each sample 20 picomoles of siRNA-FITC were mixed with 3.50 GE SPIO nanoparticles and the complexes were allowed to incubate at RT for 20 min. Media was refreshed in the plates prior to adding the siRNA/nanoparticles to the cells. The siRNA/GE SPIO complexes were added to the cells and the magnetic plate was placed in the incubator at 37° C. for 1 h or at RT for 10 min (exposure time). ˜24 h post transfection, cells were washed in PBS one time and released from the plates with Accutase® after 3 minutes of incubation at 37° C. Cells were spun down and the cell pellet was resuspended in 1500 PBS and analyzed by flow cytometry. GFP-positive cells were detected in FL1 (fluorescence channel 1, corresponding to the 525 nm wavelength) according to the protocol. Flow cytometry was performed using a Beckman Coulter FC-500™ flow cytometer. The transfection efficiency of siRNA delivery to hESC-MEL-2 cells using GE SPIO magnetic nanoparticles and magnetic field is higher at 37° C. for 60 minutes compared to the same at room temperature (25° C.) for 10 minutes, as shown in FIG. 7.

### Example 6

### Increased Viability when Using Magnetic Particles Vs Lipid Transfection in MEL-2 Human Embryonic Stem Cells

Materials:

MEL-2 human embryonic stem cells were purchased from Millipore® (cat# SCC021). Matrigel™ (cat#356234) was purchased from BD®. mTESR™1 cell culture media was purchased from STEMCELL™ Technologies (cat#05850). StemPro® Accutase® Cell Dissociation Reagent was procured from Life Technologies (cat# A1110501). Y-27632 (ROCK inhibitor) was obtained from Enzo®Life Sciences (cat# ALX-270-333-M001). BLOCK-iT™ Fluorescent Oligo (siRNA-FITC) was used to estimate the transfection efficiency (Life Technologies, cat#2013). The GE magnetic nanoparticles were generated in-house. The magnetic field exposure was performed using a commercial magnetic plate (Boca Scientific, cat# MF10000). The PolyMag nanoparticles were purchased from Boca Scientific (cat #PN30100). Propidium Iodide was purchased from Sigma Aldrich (cat# P4864).

Cells and Media:

MEL-2 human embryonic stem cells were purchased from Millipore® (cat# SCC021). Matrigel™ (cat#356234) was purchased from BD®. mTESR™1 cell culture media was purchased from STEMCELL™ Technologies (cat#05850). StemPro® Accutase® Cell Dissociation Reagent was procured from Life Technologies (cat# A1110501). Y-27632 (ROCK inhibitor) was obtained from Enzo®Life Sciences (cat# ALX-270-333-M001). BLOCK-iT™ Fluorescent Oligo (siRNA-FITC) was used to estimate the transfection efficiency (Life Technologies, cat#2013).

Delivery of siRNA-FITC in MEL-2 Human Embryonic Stem Cells Using Commercial Nanoparticles (PolyMag):

MEL-2 human embryonic stem cells were seeded in 24-well TCP coated with Matrigel. Cells were allowed to incubate for ˜24 h and reach 50-60% confluency before transfection was performed. Each sample was treated with 20 pmoles siRNA FITC. The Lipofectamine samples were prepared as follows: 20 Lipofectamine was mixed with 20 pmoles siRNA in mTESR1 media without the 5× growth factors (basal medium). The mixture was allowed to incubate at RT for 15-20 min. The lipid/siRNA complex was added to the cells and allowed to incubate overnight. The PolyMag samples were prepared according to the manufacturer's protocol. Briefly, the siRNA was diluted to 200 ul mTESR1 (basal medium) then was added to the PolyMag solution and allowed to incubate for 20 min at RT. Cells were then exposed to the commercial permanent magnet for ˜1 h at RT then cultured in a 37° C., 5% CO₂ incubator until next day.

The GE SPIO Samples were Generated as Follows: 7 μl GE nanoparticles were mixed with 20 pmoles siRNA-FITC and allowed to incubate at RT for 20 min. The GE nanoparticles/siRNA-FITC complexes were then added to the cells and allowed to incubate on the magnet for 1 h at RT. Cells were then incubated at 37° C. for overnight.

All samples were analyzed next day by flow cytometry for determining the efficiency of siRNA-FITC delivery and viability. Viability was determined by staining the cells with propidium iodide (PI) according to the following protocol. Cells were harvested and washed twice with PBS. At the end of the washing step, cells were centrifuged at 300×g for 5 min and then the buffer was decanted from the pellet cells. 50 of PI staining solution (10 ug/ml) was added to each sample. Cells were incubated in ice for 10 min. Cells were spun down and the cell pellet was resuspended in 150 μl PBS and analyzed by flow cytometry in FL1 channel (corresponding to the 525 nm wavelength) and FL-2 channel (for PI signal). The results of cell viability and transfection efficiency for siRNA delivery to hESC-MEL-2 cells using commercially available magnetic particles PolyMag, GE SPIO magnetic nanoparticles and Lipofectamine are shown in FIG. 8. The data shows viability is better for delivery of siRNA in presence of magnetic particles with 1 hour exposure to magnetic fields at 37° C. compared to using Lipofectamine (FIG. 8).

### Example 7

### Effect of Magnetic Nanoparticles Volume on Plasmid Transfection Efficiency into B35 Cells

Materials:

The B35 rat neuroblastoma cells (cat# CRL-2754) were purchased from ATCC®, Manssas, Va., USA. Neuromag particles (cat# NM50200) were purchased from Boca Scientific Boca Raton, Fla., USA. To perform the transfections a plasmid encoding maxGFP, a green fluorescent protein from the copepod Pontellina p was used from a kit available from Lonza, (cat# VSC-1001). MaxGFP stock was at 0.5 μg/μl.

Cells and Medium:

B35 rat neuroblastoma cells (ATCC®, cat# CRL-2754) were cultured in ATCC® formulated Dulbecco's Modified Eagle's Medium (DMEM, cat#30-2002) supplemented with 10% FBS according to the manufacturer's ATCC® protocol.

Plasmid Delivery Using Magnetic Nanopartciles (Neuromag):

B35 neuroblastoma cells (passage #8) were seeded in 24 well plates at 0.7×10⁵ cells/well. Cells were allowed to incubate at 37° C. for overnight and transfected 24 h post seeding at ˜60-70% confluence. Cell count was measured using Countess® Automated Cell Counter (Invitrogen) according to the manufacturer's protocol. Different wells of B35 cells within a plate received different volumes of NeuroMag nanoparticles (1.750, 3.50, and 5.250). Each condition was performed in duplicates. Each well was transfected with 1 μg plasmid GFP (0.5 μl from the stock). The transfection was performed according to Boca Scientific protocol. Briefly, the NeuroMag particles were mixed with the DNA and incubated for 15-20 min at RT. The nanoparticles/DNA complexes were then added to the cells. B35 cells were evaluated next day (˜24 h) for transfection efficiency by flow cytometry using a Beckman Coulter FC-500™ Flow Cytometer. Briefly, spent media was removed and cells were washed once in PBS at RT. After PBS removal, enzymatic release of the cells was performed using Trypsin-EDTA (ATCC® cat#30-2101) at 37° C. for 2-5 min. Cells were spun down and the cell pellet was resuspended in 1500 PBS and analyzed by flow cytometry in FL1 channel (corresponding to the 525 nm wavelength) according to the protocol. The plasmid delivery efficiency increases with the increasing magnetic particle concentration, as shown in FIG. 9.

### Example 8

### Effect of Different Field Intensities at Various Concentrations of Nanoparticles Using CHO Cells

Materials:

The Chinese Hamster Ovary (CHO) cells (cat# CCL-61) were purchased from ATCC®, Manssas, Va., USA. PolyMag particles (cat# PN30100) were purchased from Boca Scientific Boca Raton, Fla., USA. To perform the transfections a plasmid encoding maxGFP, a green fluorescent protein from the copepod Pontellina p was used from a kit available from Lonza, (cat# VSC-1001). MaxGFP stock was at 0.5 μg/μl.

Cells and Medium:

The Chinese Hamster Ovary (CHO) cells (ATCC, cat# CCL-61) were cultured in ATCC-formulated F-12K medium (ATCC, cat#30-2004) supplemented with 10% FBS according to the ATCC® protocol (F12-K complete media).

Plasmid Delivery Using Magnetic Nanopartciles (PolyMag) and an electromagnet:

CHO cells (passage#5) were seeded in 24-well plates at 0.3×10⁵ cells/well in F-12K complete media. At 24 h post seeding media spent media was removed and fresh media was added to the cells. Cells were transfected using the PolyMag nanoparticles according to the Boca Scientific protocol. Briefly, different amounts of PolyMag: 0.50 μl (the recommended volume by the manufacturer), 2 μl and 5 μl were mixed with maxGFP (0.5 ug/each well) and allowed for incubation at RT for 15 min. The DNA/nanoparticles complexes were then added to the cells. The plate was placed on the electromagnet, wherein the electromagnet used for these test creates a field of about 0.23 T at 1.8 A. The field was varied by changing the electromagnet current. At 1.2 A, the magnetic field was 0.15 T and at 0.6 A, the magnetic field was 0.07 T. Three different field strengths were used in these experiments: 0.23 T (at 1.8 A), 0.15 T (at 1.2 A) and 0.07 T (0.6 A). 24 h post transfection, cells were evaluated for transfection efficiency by flow cytometry using a Beckman Coulter FC-500™ Flow Cytometer. Briefly, spent media was removed and cells were washed once in PBS at RT. After PBS removal, enzymatic release of the cells was performed using Trypsin-EDTA (ATCC® cat#30-2101) at 37 C for 2-5 min. Cells were spun down and the cell pellet was re-suspended in 1500 PBS and analyzed by flow cytometry in FL1 channel (corresponding to the 488 nm wavelength) according to the protocol. The effects of varying magnetic particle concentration and magnetic field amplitude using an electromagnet for plasmid delivery to CHO cells were determined, as shown in FIG. 10. The graphs illustrate, at the lower electromagnetic current setting, higher particle concentration results in increased transfection efficiency; however, at higher electromagnetic current settings (higher magnetic field amplitudes), the increased particle concentration does not have much effect on transfection efficiencies.

### Example 9

### Effect on Plasmid DNA Transfection into HEK293 Using Commercial Nanoparticles with and without a Permanent Magnet

Materials Used:

Human embryonic kidney (HEK293) cells were purchased from ATCC®, Manssas, Va., USA (cat# CRL-1573). CombiMag particles were purchased from Boca Scientific Boca Raton, Fla., USA (cat# CM20100). Lipofectamine® 2000 transfection reagent was purchased from Invitrogen® (cat#11668019). To perform the transfections a plasmid encoding maxGFP, a green fluorescent protein from the copepod Pontellina p was used from a kit available from Lonza, (cat# VSC-1001). pMaxGFP stock was at 0.5 μg/μl. The magnetic field exposure was performed using a commercial magnetic plate (Boca Scientific, cat# MF10000).

Cells and Medium:

HEK 293 cells were cultured in ATCC-formulated Eagle's Minimum Essential Medium, (catalog #30-2003) supplemented with 10% FBS. Cells were cultured according to the manufacturer's protocol.

Plasmid Transfection in HEK293 Using Commercial Nanoparticles with and without the Magnet Exposure:

HEK 293 cells were seeded in 24-well TCP at 0.7×10̂5 cells/well in the recommended media. Cells were allowed to incubate at 37° C. and 5% CO2 overnight and transfected 24 h post seeding at ˜60-70% confluence. Cell count was measured using Countess® Automated Cell Counter (Invitrogen) according to the manufacturer's protocol. Each well was transfected with 0.5 μg pGFP. All experimental samples were transfected with 1 μl CombiMag and 2 ul Lipofectamine® per sample according to the following protocol: 1 μl CombiMag was first mixed with 0.50 pGFP and allowed to incubate for few minutes. 20 Lipofectamine®2000 was added to the CombiMag/DNA mix and allowed to further incubate at RT for 30 min. The DNA/Lipid/CombiMag complexes were added to the HEK293 cells and allowed to incubate with the cells directly (no magnet) or placed on the commercial magnet (plus magnet) for another 20 min. All samples were incubated overnight at 37° C. in the 5% CO₂ incubator for ˜24 h. Transfection was evaluated 24 h later by flow cytometry using a Beckman Coulter FC500 Flow Cytometer. Briefly, spent media was removed and cells were washed once in PBS at RT. After PBS removal, enzymatic release of the cells was performed using Trypsin-EDTA (ATCC® cat#30-2101) at 37 C for 2-5 min. Cells were spun down and the cell pellet was resuspended in 1500 PBS and analyzed by flow cytometry in FL1 channel (corresponding to the 525 nm wavelength) according to the protocol. The transfection efficiency measured for the cells with magnet exposure shows similar efficiency compared to cells not exposed to magnet, as shown in FIG. 11.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A system for optimizing introduction of a nucleic acid into a cell using magnetic delivery, wherein the system comprises:
a) a control module;
b) an incubation module for incubating magnetic nanoparticles and nucleic acids; and
c) a transfection module.

2. The system of claim 1, further comprising an analysis module.

3. The system of claim 1, wherein the system is a microfluidic system.

4. The system of claim 1, wherein the control module comprises control software, and wherein the control software selects experimental conditions to be analyzed for optimizing the magnetic delivery of the nucleic acid.

5. The system of claim 1, wherein the magnetic nanoparticles and nucleic acids incubation module comprises a plurality of chambers in which magnetic nanoparticles with at least one different property are placed in separate chambers.

6. The system of claim 3, wherein the different property of the magnetic particles in the separate chambers is selected from the group consisting of concentration of the magnetic nanoparticles, size of the magnetic nanoparticles, composition of the magnetic nanoparticles and surface properties of the magnetic nanoparticles.

7. The system of claim 1, wherein the magnetic nanoparticle and nucleic acid incubation module further comprises a plurality of chambers in which nucleic acids with at least one different property are placed in separate chambers.

8. The system of claim 5, wherein the different property of the nucleic acids is selected from the group consisting of nucleotide sequence of the nucleic acids, concentration of the nucleic acids, pre-incubation of nucleic acids with additional chemical agents to create nucleic acid complexes with the desired electric charge properties, and number of nucleotide bases in the nucleic acids.

9. The system of claim 1, wherein the system further comprises a particle incubation reactor, wherein the magnetic nanoparticles, nucleic acids or nucleic acid complexes are mixed and incubated for varying times to promote formation of nucleic acid/magnetic nanoparticle complexes.

10. The system of claim 9, wherein the particle incubation reactor further comprises a platform that permits agitation of the magnetic nanoparticles and nucleic acids during the formation of the nucleic acid/magnetic nanoparticle complexes.

11. The system of claim 9, wherein the particle incubation reactor further comprises a platform that regulates temperature during the formation of the nucleic acid/magnetic nanoparticle complexes.

12. The system of claim 1, wherein the transfection module comprises a nucleic acid transfection reactor that comprises a plurality of chambers in which cells are mixed and incubated with the nucleic acid/magnetic nanoparticle complexes and exposed to a magnetic field for varying times, at varying magnetic field strengths, and at varying magnetic gradients.

13. The system of claim 12, wherein the nucleic acid transfection reactor further comprises a platform that permits agitation of the magnetic nanoparticles and nucleic acids during incubation of the cells with the nucleic acid/magnetic nanoparticle complexes.

14. The system of claim 1, wherein the analysis module provides a method for determination of transfection efficiency for each combination of experimental conditions tested for the introduction of nucleic acids into the cells.

15. The system of claim 14, wherein the analysis module further provides a method for determination of cell viability for each combination of experimental conditions tested for the introduction of nucleic acids into the cells.

16. The system of claim 15, wherein the transfection efficiency and the cell viability data obtained for each set of experimental conditions tested for the introduction of the nucleic acids into the cells is reported to the control software to provide to a user an optimal combination of experimental conditions for introduction of nucleic acids into the cells.

17. The system of claim 16, wherein a range of the experimental conditions is provided to the user for the optimal combination of experimental conditions for introduction of nucleic acids into the cells.

18. The system of 17, wherein the system runs in batch production mode.

19. The system of 18, wherein the system applies an optimal combination of experimental conditions for introduction of nucleic acids into the cells determined by the control software repeatedly and in parallel over a plurality of nucleic acid transfection reactors to increase the throughput of transfected cells.

20. The system of claim 1, wherein operation of the system is partially or completely automated.

21. The system of claim 1, further comprising a detector for measuring percent knockdown of one or more targeted genes.

22. The system of claim 1, further comprising a detector for measuring copy number of the transfected nucleic acids.

23. The system of claim 1, further comprising a detector for measuring expression level of the delivered nucleic acids.

24. The system of claim 1, further comprising a temperature sensor and set up arrangement for probing required temperature selecting from multiple temperatures

25. The system of claim 1, wherein the system is a high throughput system.

26. The system of claim 1, wherein the nucleic acid is DNA or RNA.

27. The system of claim 1, wherein the nucleic acid is DNA.

28. A system for optimizing introduction of a nucleic acid into a cell using magnetic delivery, wherein the system comprises:
a) a control module;
b) an incubation module for incubating magnetic nanoparticles and nucleic acids;
c) a transfection module; and
d) an analysis module.
